(19) United States
(12) Patent Application Publication
(10) Pub. No.: US 2023/0000894 A1
(43) Pub. Date: Jan. 5, 2023

(54) TREATING MITOCHONDRIAL DNA DEPLETION DISORDERS

(71) Applicant: MITORAINBOW THERAPEUTICS, INC., Cary, NC (US)

(72) Inventors: Curtis Lianjie CUI, Cary, NC (US); Ward PETERSON, Cary, NC (US); Benjamin Robinson YERXA, Cary, NC (US)

(21) Appl. No.: 17/640,378

(22) PCT Filed: Sep. 4, 2020
(86) PCT No.: PCT/US2020/049413

§ 371 Date: Mar. 4, 2022

Related U.S. Application Data
(60) Provisional application No. 62/896,218, filed on Sep. 5, 2019.

(51) Int. Cl. A61K 31/7084; A61K 9/00; A61P 21/00

(52) U.S. Cl. CPC A61K 31/7084; A61K 9/0019; A61P 21/00

(57) ABSTRACT

The present disclosure describes a method for treating mitochondrial DNA depletion syndrome by administration of a therapeutic amount of a composition comprising a dinucleotide compound or a mixture thereof. Further described herein are compounds, compositions and methods for the treatment of TK2 deficiency.

### CROSS REFERENCE TO RELATED APPLICATIONS

This application claims the benefit of U.S. Provisional Patent Application Ser. No. 62/896,218, filed Sep. 5, 2019, the disclosure of which is incorporated, in its entirety, by this reference.

### FIELD OF THE INVENTION

The present disclosure describes a method for treating mitochondrial DNA depletion syndrome (MDS) by administration of a therapeutic amount of a composition comprising a multinucleotide compound or a mixture thereof. Further described herein are compounds, compositions and methods for the treatment of thymidine kinase 2 (TK2) deficiency.

### BACKGROUND

Mitochondria are known as the energy producing organelles of cells. These double membraned organelles function independently of the cells in which they reside and even have their own genome, separate from the nuclear genome. While the nuclear genome encodes for some mitochondrial proteins, the mitochondrial genome specifically encodes for proteins associated with energy associated metabolic processes, including the electron transport chain and ATP production. Mitochondrial DNA depletion syndrome (MDS) is an umbrella term used to describe a wide range of disorders all characterized by highly reduced cellular mitochondrial DNA. MDS is the result of mutations in nuclear genes involved in nucleotide synthesis or replication of mitochondrial DNA, and have been associated with a variety of genes including TK2, SUCLA2, SUCLG1, POLG, DGUOK, MPV17, TYMP, and RRM2B. (Chansprasert et al., 2017). As a result, these autosomal recessive disorders result in extreme depletion of mitochondrial DNA (mtDNA) which in turn negatively impacts energy production. (El-Hattab et al., 2013).

Symptoms appear in infancy or early childhood and primarily impact the tissues of the muscles, liver, or brain and generally present as muscle weakness, organ and neurological dysfunction, and weight loss. However, these disorders are phenotypically heterogeneous. For example, mutations in SUCLA2, SUCLG1, or RRM2B result in encephalomyopathic MDS and clinically present as hypotonia and neurological issues. Mutations in DGUOK, MPV17, or POLG present as early-onset liver dysfunction. (El-Hattab et al., 2013). Mutations in TYMP are associated with progressive gastrointestinal dysmotility and peripheral neuropathy. (El-Hattab et al., 2013). The severity and progression of these disorders is also highly variable, from mild manifestations to severe symptoms that result in death during infancy and childhood. Because of such heterogeneity in clinical manifestations, treatment options are limited to managing symptoms primarily through nutritional supplementation.

Thymidine kinase 2 (TK2) deficiency is a specific type of MDS. The enzyme thymidine kinase 2 is a nuclear encoded enzyme involved in mitochondrial DNA (mtDNA) synthesis involved in the recycling of nucleotides. (Genetics Home Reference, 2013). Mutations in the TK2 gene reduce enzyme activity and impair mtDNA nucleotide recycling, resulting in low levels of deoxythymidine monophosphate and deoxycytidine monophosphate. This shortage in the nucleotide pool impacts mtDNA synthesis and ultimately results in progressive myopathy that may begin in early childhood, eventually resulting in loss of motor skills. (Garone et al., 2018).

Currently, no disease modifying therapies exist for TK2 disease and treatment is primarily supportive. Although oral supplementation of deoxythymidine monophosphate, (dTMP) and deoxycytidine monophosphate (dCMP) has demonstrated the ability to restore mtDNA depletion in animal models and increase overall survival, such treatments require high doses of each of the deoxyribonucleoside monophosphates (dNMP) in order to achieve an overall therapeutic benefit with significant side effects.

### SUMMARY

One embodiment described herein is a method of treating mitochondrial DNA depletion syndrome in a subject in need thereof comprising administering to the subject a therapeutically effective amount of a multinucleotide composition comprising the compound of Formula I

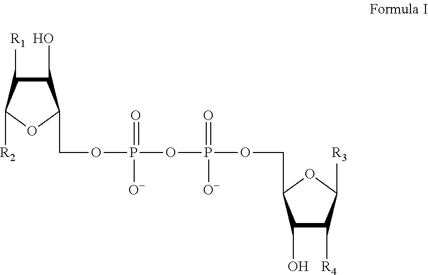

wherein:

### R₁ is H or OH;

R₂ is a purine derivative or pyrimidine derivative;
R₃ is a purine derivative or pyrimidine derivative; and

### R₄ is H or OH.

In one aspect, the composition comprises the compound of Formula I wherein R₁ is H, R₂ is 5-methyl-2H-1λ2-pyrimidine-2,4(3H)-dione, R₃ is 4-amino-2H-1λ2-pyrimidine-2-one, and R₄ is OH. In another aspect, the composition is a mixture further comprising the compound of Formula I, wherein R₁ is OH, R₂ is 9λ2-purine-6-amine, R₃ is 2-amino-9λ2-purin-6(1 H)-one, and R₄ is OH.

In one aspect, the composition is administered orally, enterically, intravenously, or subcutaneously. In a further aspect, the composition is administered intravenously.

In one aspect, blood plasma levels of each mononucleoside is higher after intravenous administration as compared to blood plasma levels after oral administration of individual mononucleotides.

In another aspect, intravenous administration of the composition results in a blood plasma level having a first AUC₀₋₂₄ₕ and oral administration of individual mononucleotides results in blood plasma levels having a second AUC₀₋₂₄ₕ, and wherein the ratio of the first AUC₀₋₂₄ₕ to the second AUC₀₋₂₄ₕ is between about 100 to about 400.

In yet another aspect, when the composition is administered at a dosage of between about 1 mg/kg to about 20 mg/kg, blood plasma concentration of the corresponding nucleosides is between about 50 ng/ml to about to about 5000 ng/ml.

In one aspect, the mitochondrial DNA depletion syndrome comprises thymidine kinase 2 deficiency, succinyl CoA synthetase deficiency, deoxyguanosine kinase deficiency, succinyl CoA ligase deficiency, ribonucleotide-diphosphate reductase subunit M2 B enzyme deficiency, thymidine phosphorylase deficiency, and polymerase gamma deficiency. In a further aspect, the mitochondrial DNA depletion syndrome comprises thymidine kinase 2 deficiency.

In one aspect, the subject is a human.

Another embodiment described herein is a method for the treatment of thymidine kinase 2 (TK2) deficiency in a subject in need thereof comprising: a) obtaining a nucleic acid sample from a subject; b) determining if the subject has TK2 deficiency; c) administering a therapeutically effective amount of a composition comprising a multinucleotide compound of Formula 1:

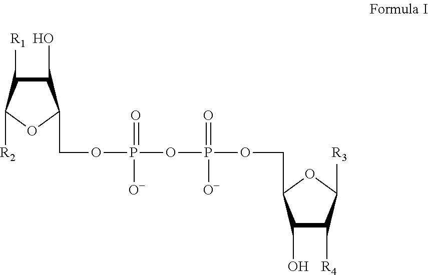

wherein:

### R₁ is H or OH;

R₂ is a purine derivative or pyrimidine derivative;
R₃ is a purine derivative or pyrimidine derivative; and
R₄ is H or OH; and d) measuring blood plasma levels of corresponding mononucleosides; wherein the blood plasma levels of corresponding nucleosides is between about 50 ng/mL to about 5000 ng/mL.

Another embodiment described herein is a compound of Formula I:

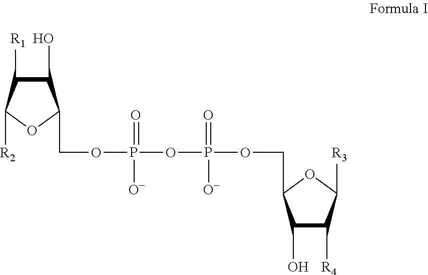

wherein

### R₁ is H or OH;

R₂ is a purine derivative or pyrimidine derivative;
R₃ is a purine derivative or pyrimidine derivative; and

### R₄ is H or OH.

In one aspect, the compound is substantially free from impurities. In another aspect, R₁ is H, R₂ is thymine and R₃ is cytosine, R₄ is OH.

In another aspect, a pharmaceutical composition comprises at least one pharmaceutically acceptable excipient and a therapeutically effective amount of any of the compounds described herein. In a further aspect, the composition comprises the compound wherein R₁ is H, R₂ is 5-methyl-2H-1λ2-pyrimidine-2,4(3H)-dione, R₃ is 4-amino-2H-1λ2-pyrimidine-2-one, and R₄ is OH.

One or more aspects and embodiments may be incorporated in a different embodiment although not specifically described. That is, all aspects and embodiments may be combined in any way or combination.

### BRIEF DESCRIPTION OF THE DRAWINGS

FIG. 1 depicts the structure and metabolism of test articles.

FIG. 2 depicts plasma levels of dT and dC up to 48 h post-dose, corrected for the amount of labeled vs unlabeled test articles and normalized to the total dose of test article administered.

FIG. 3 depicts AUC₀₋₂₄ₕ exposure of label-corrected and dose-normalized total dT and dC following acute P.O. administration of labeled and unlabeled dCMP+dTMP, and acute I.V. administration of labeled and unlabeled dC(P₂)dT. The ratios of plasma AUC₀₋₂₄ₕ for I.V. vs P.O. administrations were approximately 350- and 110-fold higher for dT and dC, respectively.

FIG. 4 depicts plasma levels of dT and dC up to 48 h following a single I.V. administration and one and two S.C. administrations of labeled and unlabeled dC(P₂)dT.

FIG. 5 depicts AUC₀₋₂₄ₕ exposure of label-corrected total dT and dC following parenteral administration of dC(P₂)dT.

### DETAILED DESCRIPTION

The present disclosure describes a method of treating mitochondrial DNA depletion syndrome (MDS). Also described herein are compounds and methods for the treatment of thymidine kinase 2 (TK2) deficiency.

### Definitions

The term “about” as used herein refers to any values, including both integers and fractional components that are within a variation of up to ±10% of the value modified by the term “about.” For example, the phrase “about 50%” is equivalent to any vale ≈50±10%, e.g., 44.6%, 45%, 46%, 47%, 48%, 49%, 49.5%, 50%, 50.3%, 51%, 52%, 53%, 54%, 55%, inter alia.

As used herein, “a” or “an” means one or more unless otherwise specified. As used herein, “effective amount” refers to the amount sufficient to achieve a therapeutic effect when administered to a patient in need of treatment.

The term “AUC_{0→24}” as used herein refers to the area under the blood (plasma, serum, or whole blood) concentration versus time curve from 0 to 24 hours.

The term “derivate” as used herein refers to a compound derived from purine or pyrimidine, including a compound formed from a purine or pyrimidine precursor, respectively.

The term “dosage” or “dose” denote any form of the active ingredient formulation that contains an amount sufficient to produce a therapeutic effect with a single administration. The dosage form used herein may be for oral, enteric or intravenous administration.

The term “formulation” or “composition” as used herein refers to the active pharmaceutical ingredient or drug in combination with pharmaceutically acceptable excipients. This includes orally administrable formulations as well as formulations administrable by other means.

Terms such as “include,” “including,” “contain,” “containing,” “has,” or “having,” and the like, mean “comprising.”

The term “or” can be conjunctive or disjunctive.

The term “mononucleoside” as used herein refers to a single purine or pyrimidine base linked to a sugar. Examples include deoxycytidine (dC) and deoxythymidine (dT).

The term “mononucleotide” as used herein refers to a single purine or pyrimidine base linked to a sugar and a phosphate group in free acid or any salt form such as disodium salt. Examples include deoxycytidine monophosphate (dCMP) and deoxythymidine monophosphate (dTMP).

The term “dinucleotide” as used herein refers to a compound containing two mononucleotides in free acid or any salt form such as disodium salt. For example, deoxycytidine-deoxythymidine diphosphate dC(P₂)dT or dC(P₂)dT.Na₂.

The term “multinucleotide” as used herein refers to a compound containing more than one mononucleotide. For example, a multinucleotide compound may contain two nucleotides. The mononucleotides that make up a multinucleotide may all be identical or different.

The term “corresponding mononucleoside” as used herein refers to the single mononucleoside corresponding to a mononucleotide, or the single mononucleoside component corresponding to a dinucleotide or multinucleotide. For example, in the mononucleotide deoxycytidine monophosphate (dCMP), the corresponding mononucleoside is deoxycytidine (dC). In another example, the dinucleotide deoxycytidine-deoxythymidine diphosphate (dC(P₂)dT), the corresponding mononucleosides are deoxycytidine (dC) and deoxythymidine (dT).

As used herein, the terms “subject” and “patient” are used interchangeably herein. In one embodiment, the subject is a human.

The term “substantially pure” as used herein means having a level of purity that would be recognized as “pure” by those of skill in the art. This level of purity may be less than 100%.

When referring to the compounds disclosed herein, the following terms have the following meanings unless indicated otherwise. The following definitions are meant to clarify, but not limit, the terms defined. If a particular term used herein is not specifically defined, such term should not be considered indefinite. Rather, terms are used within their accepted meanings.

As used herein the term “purine” refers to a heterocyclic aromatic organic compound that consists of a pyrimidine ring fused to an imidazole ring.

As used herein the term “pyrimidine” refers to an aromatic heterocyclic organic compound.

As used herein “substituted” refers to a substitution of a hydrogen atom, which would otherwise be present on the substituent. When discussing ring systems, the optional substitution is typically with 1, 2, or 3 substituents replacing the normally-present hydrogen. When referencing straight and branched moieties, however, the number of substitutions can be more, occurring wherever hydrogen is usually present. The substitutions can be the same or different. Illustrative substitutions include nitro, —NR′R″, cyano, —NR′COR″′, alkyl, alkenyl, —C(O), —SO₂R′″, —NR′SO₂R′″, —SO₂NR′R″, —CONR′R″, —CONHC₆H₅, hydroxy, alkoxy, alkylsulfonyl, haloalkyl, haloalkenyl, haloalkoxy, mercapto (—SH), thioalkyl, halogen, cycloalkyl, heterocyclyl, aryl, or heteroaryl, where R′ and R″ are the same or different and each represents hydrogen or alkyl; or when R′ and R″ are each attached to a nitrogen atom, they may form a saturated or unsaturated heterocyclic ring containing from 4 to 6 ring atoms, and wherein R′″ is alkyl or haloalkyl.

In certain cases, the depicted substituents can contribute to optical and/or stereoisomerism. Compounds having the same molecular formula but differing in the nature or sequence of bonding of their atoms or in the arrangement of their atoms in space are termed “isomers.” Isomers that differ in the arrangement of their atoms in space are termed “stereoisomers.” Stereoisomers that are not mirror images of one another are termed “diastereomers” and those that are non-superimposable mirror images of each other are termed “enantiomers”. When a compound has an asymmetric center, for example when it is bonded to four different groups, a pair of enantiomers is possible. An enantiomer can be characterized by the absolute configuration of its asymmetric center and is designated (R) or (S) according to the rules of Cahn and Prelog (Cahn et al., 1966, Angew. Chem. 78: 413-447, Angew. Chem., Int. Ed. Engl. 5: 385-414 (errata: Angew. Chem., Int. Ed. Engl. 5:511); Prelog and Helmchen, 1982, Angew. Chem. 94: 614-631, Angew. Chem. Internat. Ed. Eng. 21: 567-583; Mata and Lobo, 1993, Tetrahedron: Asymmetry 4: 657-668) or can be characterized by the manner in which the molecule rotates the plane of polarized light and is designated dextrorotatory or levorotatory (namely, as (+)- or (−)-isomers, respectively). A chiral compound can exist as either an individual enantiomer or as a mixture thereof. A mixture containing equal proportions of enantiomers is called a “racemic mixture”.

In certain embodiments, the compounds disclosed herein can possess one or more asymmetric centers; and such compounds can therefore be produced as the individual (R)- or (S)-enantiomer or as a mixture thereof. Unless indicated otherwise, for example by designation of stereochemistry at any position of a formula, the description or naming of a particular compound in the specification and claims is intended to include both individual enantiomers and mixtures, racemic or otherwise, thereof. Methods for determination of stereochemistry and separation of stereoisomers are well-known in the art. In particular embodiments, stereoisomers of the compounds provided herein are depicted upon treatment with base.

In certain embodiments, the compounds disclosed herein are “stereochemically pure”. A stereochemically pure compound has a level of stereochemical purity that would be recognized as “pure” by those of skill in the art. Of course, this level of purity may be less than 100%. In certain embodiments, “stereochemically pure” designates a compound that is substantially free, i.e. at least about 85% or more, of alternate isomers. In particular embodiments, the compound is at least about 85%, about 90%, about 91%, about 92%, about 93%, about 94%, about 95%, about 96%, about 97%, about 98%, about 99%, about 99.5% or about 99.9% free of other isomers.

In addition, a pharmaceutically acceptable prodrug of the compound represented by the Formula I is also included in the present invention. The pharmaceutically acceptable prodrug refers to a compound having a group which can be converted into an amino group, a hydroxyl group, a carboxyl group, or the like, by solvolysis or under a physiological condition. Examples of the groups forming the prodrug include those as described in Prog. Med., 5, 2157-2161 (1985) or “Pharmaceutical Research and Development” (Hirokawa Publishing Company, 1990), vol. 7, Drug Design, 163-198. The term prodrug is used throughout the specification to describe any pharmaceutically acceptable form of a compound which, upon administration to a patient, provides the active compound. Pharmaceutically acceptable prodrugs refer to a compound that is metabolized, for example hydrolyzed or oxidized, in the host to form the compound of the present invention. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound.

As used herein “pharmaceutically acceptable salt” refers to any salt of a compound disclosed herein which retains its biological properties and which is not toxic or otherwise undesirable for pesticidal, veterinary, or pharmaceutical use. Such salts may be derived from a variety of organic and inorganic counter-ions known in the art. Such salts include: (1) acid addition salts formed with organic or inorganic acids such as hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid, and like acids.

Salts further include, by way of example only, salts of non-toxic organic or inorganic acids, such as halides, such as , chloride and bromide, sulfate, phosphate, sulfamate, nitrate, acetate, trifluoroacetate, trichloroacetate, propionate, hexanoate, cyclopentylpropionate, glycolate, glutarate, pyruvate, lactate, malonate, succinate, sorbate, ascorbate, malate, maleate, fumarate, tartarate, citrate, benzoate, 3-(4-hydroxybenzoyl)benzoate, picrate, cinnamate, mandelate, phthalate, laurate, methanesulfonate (mesylate), ethanesulfonate, 1,2-ethane-disulfonate, 2-hydroxyethanesulfonate, benzenesulfonate (besylate), 4-chlorobenzenesulfonate, 2-naphthalenesulfonate, 4-toluenesulfonate, camphorate, camphorsulfonate, 4-methylbicyclo[2.2.2]-oct-2-ene-1-carboxylate, glucoheptonate, 3-phenylpropionate, trimethylacetate, tert-butylacetate, lauryl sulfate, gluconate, benzoate, glutamate, hydroxynaphthoate, salicylate, stearate, cyclohexylsulfamate, quinate, muconate, and the like.

The present disclosure includes all pharmaceutically acceptable isotopically-labelled compounds of the invention wherein one or more atoms are replaced by atoms having the same atomic number, but an atomic mass or mass number different from the atomic mass or mass number usually found in nature. Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷Oand ¹⁸O, phosphorus, such as ³²P, and sulfur, such as ³⁵S. Certain isotopically-labelled compounds of the invention, such as those incorporating a radioactive isotope, may be useful in drug or substrate tissue distribution studies. The radioactive isotopes tritium, i.e. ³H, and carbon-14, i.e. ¹⁴C, are particularly useful for this purpose in view of their ease of incorporation and ready means of detection. Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of the invention can generally be prepared by conventional techniques known to those skilled in the art or by processes analogous to those described in the accompanying Examples and Preparations using an appropriate isotopically-labeled reagents in place of the non-labeled reagent previously employed.

The present disclosure provides methods of treating mitochondrial DNA depletion syndrome. Mitochondrial DNA depletion syndrome results when mtDNA levels are extremely depleted and negatively impact cellular energy production. (El-Hattab et al., 2013). Thus, without intending to be bound by any particular theory, the compounds provided herein are thought to metabolize into their corresponding mononucleosides, and thus replenish the nucleoside pools required to provide the building blocks for mtDNA synthesis. Compounds contemplated by the disclosure include, but are not limited to, the exemplary compounds provided herein and salts thereof.

One embodiment described herein is a method for treating mitochondrial DNA depletion syndrome comprising administering a therapeutically effective amount of a multinucleotide composition comprising the compound of Formula I, or a pharmaceutically acceptable salt thereof:

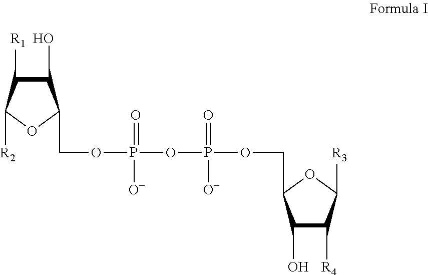

wherein

### R₁ is H or OH;

R₂ is a purine derivative or pyrimidine derivative;
R₃ is a purine derivative or pyrimidine derivative; and

### R₄ is H or OH.

Examples of purine groups include but are not limited to adenine, guanine, hypoxanthine, xanthine, theobromine, caffeine, uric acid, isoguanine. In one aspect, the purine is adenine or guanine.

Examples of pyrimidine groups include but are not limited to thymine, cytosine and uracil. In one aspect the pyrimidine is thymine or cytosine.

The multinucleotide composition may comprise more than one mononucleotide. In one aspect, the multinucleotide is a dinucleotide.

In one aspect, the multinucleotide composition comprises the compound of formula I wherein R₁ is H, R₂ is 5-methyl-2H-1λ2-pyrimidine-2,4(3H)-dione, R₃ is 4-amino-2H-1λ2-pyrimidine-2-one, and R₄ is OH. In another aspect, the composition comprises a mixture further comprising the compound of formula I, wherein R₁ is OH, R₂ is 9λ2-purine-6-amine, R₃ is 2-amino-9λ2-purin-6(1H)-one, and R₄ is OH.

In one aspect, the compound is substantially free from impurities. A substantially pure compound has a level of purity that would be recognized as “pure” by those of skill in the art. Of course, this level of purity may be less than 100%. In certain aspect, “substantially pure” designates a compound that is substantially free, i.e. at least about 85% or more, of other compounds. In particular embodiments, the compound is at least about 85%, about 90%, about 91%, about 92%, about 93%, about 94%, about 95%, about 96%, about 97%, about 98%, about 99%, about 99.5% or about 99.9% free of other compounds.

In one aspect described herein is a composition comprising one or more of the compounds of Formula I as described herein and one or more pharmaceutically acceptable carriers.

The term “composition” as used herein is intended to encompass a product comprising specific ingredients in specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

The pharmaceutical compositions for the administration of the compounds of this disclosure may conveniently be presented in unit dosage form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases.

The pharmaceutical compositions containing the active ingredient may be in a form suitable for oral use, for example, as tablets, troches, lozenges, aqueous or oily suspensions, dispersible powders or granules, emulsions and self-emulsifications as described in U.S. Pat. No. 6,451,339, hard or soft capsules, or syrups or elixirs. Compositions intended for oral use may be prepared according to any method known to the art for the manufacture of pharmaceutical compositions. Such compositions may contain one or more agents selected from sweetening agents, flavoring agents, coloring agents and preserving agents in order to provide pharmaceutically elegant and palatable preparations. Tablets contain the active ingredient in admixture with other non-toxic pharmaceutically acceptable excipients which are suitable for the manufacture of tablets. These excipients may be, for example, inert diluents such as cellulose, silicon dioxide, aluminum oxide, calcium carbonate, sodium carbonate, glucose, mannitol, sorbitol, lactose, calcium phosphate or sodium phosphate; granulating and disintegrating agents, for example, corn starch, or alginic acid; binding agents, for example PVP, cellulose, PEG, starch, gelatin or acacia, and lubricating agents, for example magnesium stearate, stearic acid or talc. The tablets may be uncoated or they may be coated enterically or otherwise by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil. Additionally, emulsions can be prepared with a non-water miscible ingredient such as oils and stabilized with surfactants such as mono-diglycerides, PEG esters and the like. Additionally, the multinucleotides described herein may be formulated as prodrugs such that the compounds are synthesized in way to allow for direct intracellular delivery of the multinucleotide to the cell.

Aqueous suspensions contain the active materials in admixture with excipients suitable for the manufacture of aqueous suspensions, such as saline salts or buffers. Such excipients are suspending agents, for example sodium carboxymethylcellulose, methylcellulose, hydroxypropylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n propyl, p hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the disclosure may also be in the form of oil in water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative. and flavoring and coloring agents. Oral solutions can be prepared in combination with, for example, cyclodextrin, PEG and surfactants.

In one aspect, the composition may be administered intravenously. The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, for example as a solution in 1,3 butane diol. Among the acceptable vehicles and solvents that may be employed are water, Ringer's solution and isotonic sodium chloride solution. In addition, sterile, axed oils are conventionally employed as a solvent or suspending medium. For this purpose any bland fixed oil may be employed including synthetic mono or diglycerides. In addition, fatty acids such as oleic acid find use in the preparation of injectables.

As demonstrated by the examples, intravenous administration of the composition as described herein, at significantly lower dosages as compared to oral administration, demonstrated improved blood plasma concentrations of the corresponding mononucleosides as compared to oral administration of individual mononucleotide therapy (Example 1).

In another aspect, the composition may be administered sub-cutaneously.

The compounds of the present disclosure may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

Dosage forms may contain between about 1 mg/kg to about 20 mg/kg of the multinucleotide composition described herein. In one aspect, the composition is administered at a dosage of between about 1 mg/kg to about 5 mg/kg. In another aspect, the composition is administered at a dosage of between about 3 mg/kg to about 8 mg/kg. In another aspect, the composition is administered at a dosage of between about 5 mg/kg to about 10 mg/kg. In another aspect, the composition is administered at a dosage of between about 8 mg/kg to about 13 mg/kg. In one aspect, the composition is administered at a dosage of between about 10 mg/kg to about 15 mg/kg. In yet another aspect, the composition is administered at a dosage of between about 13 mg/kg to about 18 mg/kg. In one aspect, the composition is administered at a dosage of between about 15 mg/kg to about 20 mg/kg.

The compounds of Formula I are useful for treating mitochondrial DNA depletion syndrome. Examples of such syndromes include, but are not limited to thymidine kinase 2 deficiency, succinyl CoA synthetase deficiency, deoxyguanosine kinase deficiency, succinyl CoA ligase deficiency, ribonucleotide-disphosphate reductase subunit M2 B enzyme deficiency, thymidine phosphorylase deficiency, and polymerase gamma deficiency. In one aspect described herein, the mitochondrial DNA depletion syndrome is thymidine kinase 2 deficiency.

The compounds of Formula I may be combined with one or more agents having the same sphere of activity, for example, to increase activity, or with substances having another sphere of activity, for example, to broaden the range of activity. Any of the individually listed agents may be used in combination with compounds of Formula I along with any other one or more listed agents independently.

Suitable agents for combination therapy include, whereby one or more compounds of Formula I may be employed as such or in the form of their preparations or formulations as combinations with one or more other pharmaceutically active substances, such as, for example, therapeutic agents for treating the symptoms of the particular form of MDS, inhibitors of ubiquitous nucleoside catabolic enzymes, including but not limited to tetrahydrouridine, triacetyluridine, N-acetylcysteine, N-acetylcysteine amide, vitamin E, immucillin H, and tipiraci. The combinations may be part of the same formulation or may be administered separately or sequentially.

A pharmaceutical preparation comprising a compound of Formula I or pharmaceutically acceptable salt thereof for delivery to a human or other mammal, is preferably in unit dosage form, in which the preparation is subdivided into unit doses containing an appropriate quantity of the active component. The unit dosage form may be a packaged preparation containing discrete quantities of the preparation, such as packaged tablets, capsules, and powders in vials or ampoules. Also, the unit dosage form may be a capsule, tablet or injectable, or it may be an appropriate number of any of these in packaged form.

The quantity of active component in a unit dose preparation may be varied or adjusted from about 0.1 mg to about 1000 mg, according to the particular application and the potency of the active component. The composition may, if desired, also contain other compatible therapeutic agents.

In therapeutic use for the treatment of mitochondrial DNA depletion syndromes in a human or other mammal, the compounds utilized in the method of treatment are administered at an initial dosage of about 0.1 mg/kg to about 100 mg/kg per interval. Preferred intervals may be daily, weekly, semi-monthly, monthly, bi-monthly, quarterly, tri-annually, semi-annually, or annually. Dosages may be administered once a day, twice a day, three times a day or as many times as necessary as determined by the practitioner. The dosages may be varied depending on the requirements of the patient, for example, the size of the human or mammal being treated, the severity of the condition being treated, the route of administration, and the potency of the compound(s) being used. Determination of the proper dosage and route of administration for a particular situation is within the skill of the practitioner. Generally, the treatment will be initiated with smaller dosages which are less than the optimum dose of the compound, which may be increased in small increments until the optimum effect under the particular circumstances is reached. For convenience, the total daily dosage may be divided and administered in portions during the day if desired.

To understand the metabolism of the compositions described herein, blood plasma levels of the multinucleotides metabolized to their corresponding mononucleosides were measured after administration. In one aspect described herein, the blood plasma levels of corresponding mononucleosides are higher as compared to administration of individual mononucleotides. In another aspect, blood plasma levels of corresponding nucleosides are between about 50 ng/mL to about 5000 ng/mL. In another aspect, blood plasma levels of corresponding nucleosides are between about 75 ng/mL to about 2500 ng/mL.

Area under the curve measurements for a time from zero to about 24 hours (AUC₀₋₂₄ₕ) for plasma levels are between about 5000 ng-h/mL to about 120,000 ng-h/mL for intravenous administration.

Another embodiment described herein is a method for the treatment of thymidine kinase 2 in a subject comprising obtaining a nucleic acid sample from the subject, determining if the subject has TK2 deficiency, administering a therapeutically effective amount of the a composition comprising the compound of Formula I:

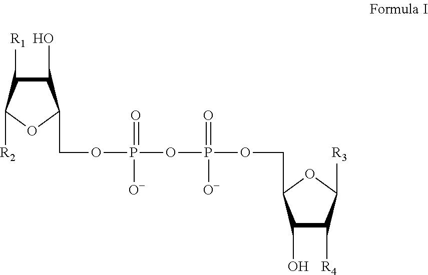

wherein

### R₁ is H or OH;

R₂ is a purine derivative or pyrimidine derivative;
R₃ is a purine derivative or pyrimidine derivative;

### R₄ is H or OH; and

measuring blood plasma levels of corresponding mononucleosides, wherein the blood plasma levels of corresponding nucleosides is between about 50 ng/mL to about 5000 ng/mL.

Patients that exhibit the phenotype TK2 deficiency including the most typical presentation of progressive muscle disease characterized by generalized hypotonia, proximal muscle weakness, loss of previously acquired motor skills, poor feeding, and respiratory difficulties, can be tested to definitively diagnose the disease.

Molecular genetic testing using a panel of genes known to cause mtDNA depletion syndrome may be performed. (Chanprasert, et al., 2012). Testing can be done by sequence analysis of the coding regions of TK2. Further tests may also be performed to confirm a TK2 deficiency diagnoses including testing serum creatine kinase concentrations, electromyography, histopathology on skeletal muscle, mitochondrial DNA content (copy number), and electron transport chain (ETC) activity in skeletal muscle.

### EXAMPLES

### Preparation of the Compounds of Formula I

The preparation of the compounds of the present disclosure or a pharmaceutically acceptable salt thereof may be accomplished may be accomplished via the route and intermediates shown herein below.

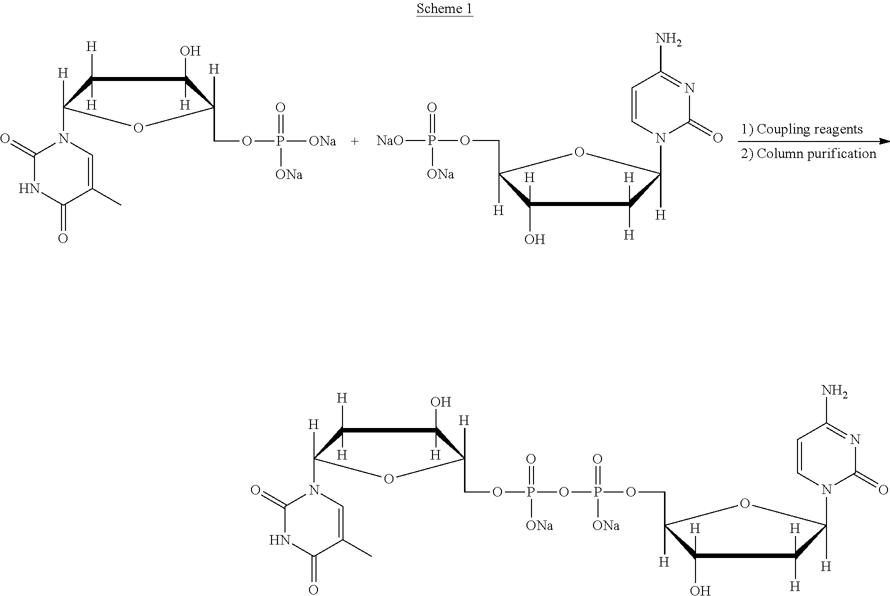

Scheme 1 describes a general process for preparing compounds of Formula I. The synthesis of the bisphosphate nucleotide was accomplished by coupling the respective nucleotide tributylammonium salts using carbonyl diimidazole as a coupling reagent to afford the desired product. In the formation of this dinucleotide asymmetrical compounds such as (dC(P₂)dC) and (dT(P₂)dT) are also formed, the current process that is being used is good enough for small scale manufacturing (1-5 g) and a better method is desirable on larger scale (100's of grams).

### Example 1

The systemic exposure of two mononucleotides (dCMP and dTMP) and their corresponding mononucleosides (dC and dT) following oral dosing of two mononucleotides (dCMP & dTMP) vs. intravenous dosing of a dinucleotide (dC(P₂)dT) was determined. Test articles were prepared as follows:

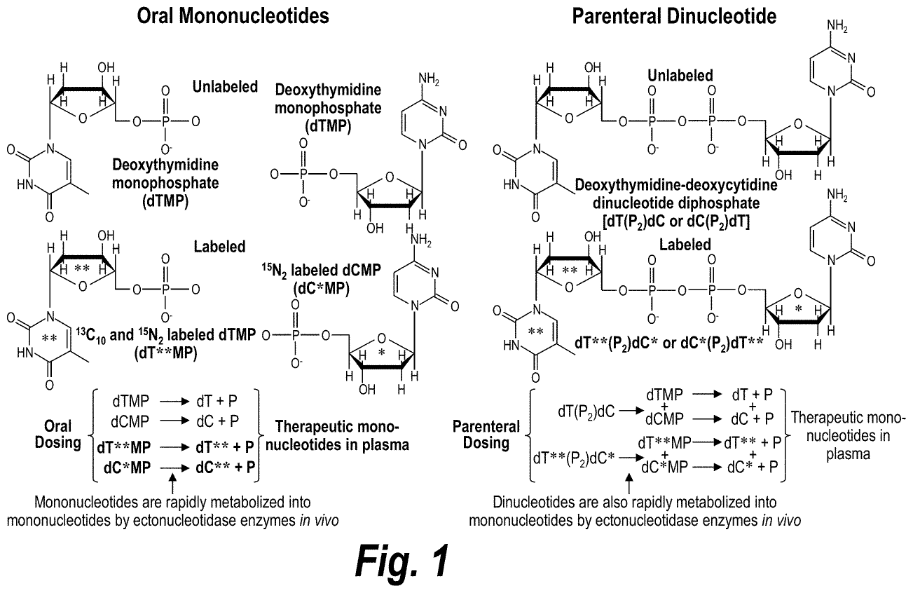

Unlabeled test articles:
dC=Deoxycytidine (nucleoside)
dT=Deoxythymidine (nucleoside)
dCMP=Deoxycytidine monophosphate (mononucleotide)
dTMP=Deoxythymidine monophosphate (mononucleotide)
dC(P₂)dT or dT(P₂)dC=Deoxycytidine-deoxythymidine diphosphate (dinucleotide)
Labeled test articles:
dC*=¹⁵N₃ labeled dC (labeled nucleoside)
dT**=¹³C10 and ¹⁵N₂ labeled dT (labeled nucleoside)
The corresponding labeled nucleotides are dC*MP and dT**MP
The corresponding labeled dinucleotide is dC*(P₂)dT** or dT**(P₂)dC*.
The structures and metabolism of these test articles are shown in FIG. 1.

The dose strength of a single IV injection of dC(P₂)dT compared to the dose strength of single P.O. administration of dCMP+dTMP was determined which resulted in comparable systemic exposure of dC and dT, where systemic exposure is quantified as AUC₀₋₂₄ₕ.

The nucleosides (dC and dT) and their corresponding monophosphate nucleotides (dCMP and dTMP) are naturally produced in the body. In order to differentiate naturally occurring vs exogenously administered dC/dT and dCMP/dTMP, a mixture of labeled dC*MP/dT**MP and unlabeled dCMP/dTMP was dosed via oral route, and a mixture of labeled dC*(P₂)dT** and unlabeled dC(P₂)dT via I.V. route. The labeled test articles served as tracers for the determination of systemic levels of exogenous dC/dT and dCMP/dTMP following dosing.

Plasma samples were then analyzed for the following analytes: dT**, dC*, dT**MP, dC*MP, and dC*(P₂)dT**. Only dT** and dC* were detectable, suggesting rapid metabolism of dT**MP, dC*MP and dC*(P₂)dT** in vivo. The results are shown in Table 1.

TABLE 1

| | | | Target | Dose | Dose | Dose | | |
|---|---|---|---|---|---|---|---|---|
| Dose | # of | Test | Dose | vol. | vol. | Cone. | | |
| Route | Animals | Article | (mg/kg) | (mL/kg) | (mL) | (mg/mL) | Blood Collections | Euthanasia |
| P.O. | 4 | dC*MP | 100 | 5 | 4 | 20 | Pre-dose and 0.25, | 48 h post- |
| | | dT**MP | | | | | 0.5 1, 2, 4, 8, 24, | dose |
| | | dCMP | 900 | | | 180 | and 48 h post-dose | |
| | | dTMP | | | | | | |
| I.V. | 4 | dC*(P₂)dT** | 2 | 1 | 0.8 | 2 | Pre-dose and 0.1, | 48 h post- |
| | | dC(P₂)dT | 8 | | | 8 | 0.25, 0.5, 1, 2, 4, 8, | dose |
| | | | | | | | 24, and 48 h post- | |
| | | | | | | | dose | |

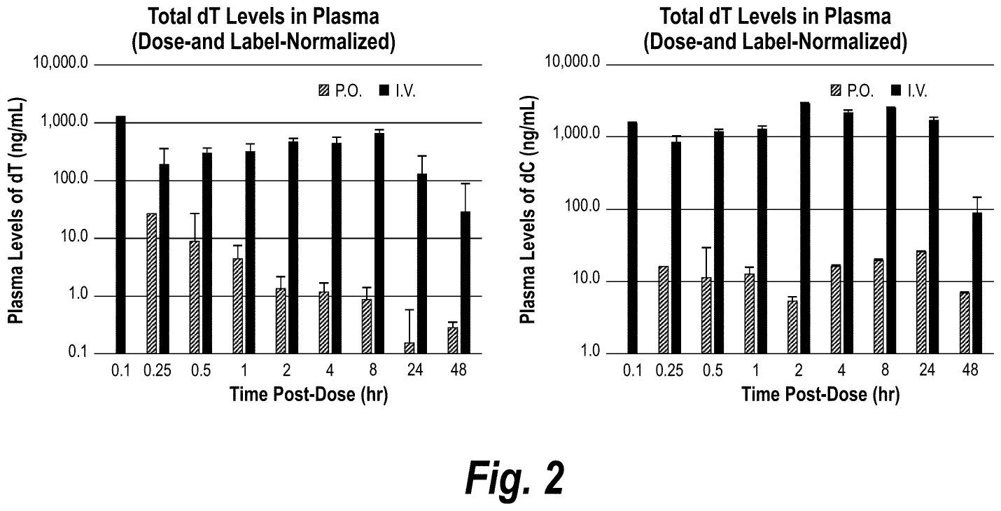

As shown in Table 1, 10% of the total test article (TA) administered by P.O. route was labeled and 20% of the total TA administered by parenteral route (I.V.) was labeled. To correct for total TA (labeled+unlabeled) administered, the plasma levels of labeled analytes were multiplied by 10 and 5 for the P.O. and I.V. treatment groups, respectively. In addition, the total TA dose administered by P.O route was 1000 mg/kg (900 mg/kg unlabeled+100 mg/kg labeled) and 10 mg/kg (8 mg/kg unlabeled and 2 mg/kg labeled) for P.O. and I.V. treatment groups, respectively. To normalize by unit dose of TA administered the plasma levels of analytes were divided by 1000 and 10 for P.O. and I.V. treatment groups, respectively. The results of the label-corrected and dose-normalized levels of dT and dC for P.O. and I.V. routes of administration are shown in FIG. 2.

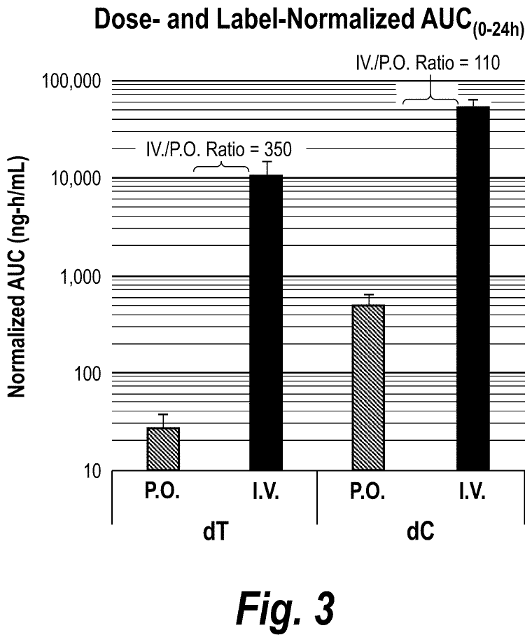

FIG. 3 shows the plasma exposures for the two analytes for the initial 24 h post-dose, determined by calculating the area-under-the-curve (AUC₀₋₂₄ₕ) for the total dose-normalized dT and dC plasma levels from FIG. 2. The differences in the AUC₀₋₂₄ₕ for I.V. vs. P.O. for both analytes are statistically different (p<0.01, t-test). The ratios of I.V./P.O. AUC₍₀₋₂₄ₕ₎'s are approximately 350 for dT and approximately for dC. Thus, based on label-corrected and dose-normalized PK analyses, these data indicate that in order to deliver comparable plasma exposure of dT and dC, we would need to administer approximately 350-fold and approximately 110-fold higher doses of oral dTMP and dCMP, respectively, compared with intravenous dC(P₂)dT.

### Example 2

The systemic exposure of dC and dT following a single I.V. injection of dC(P₂)dT vs one and two subcutaneous (S.C.) injections of dC(P₂)dT was compared, where the second S.C. injection is administered 24 h after the first. The study design is summarized in Table 2.

TABLE 2

| | | | Dose | | Total | | |
|---|---|---|---|---|---|---|---|
| | Test | # of | strength | # of | dose/rat | | |
| Route | Articles: | Rats | (mg/kg) | doses | (mg) | Blood Draws | Sacrifice |
| I.V. | dC*(P₂)T** | 4 | 2 | 1 | 0.8 | Pre and 0.1, 0.25, 0.5, | 48 hr post- |
| | dC(P₂)T | | 8 | | 3.2 | 1, 2, 4, 8, 24 and 48 hr | dose |
| S.C. | dC*(P₂)T** | 4 | 2 | 1 | 0.8 | post-dose | 48 hr post- |
| | dC(P₂)T | | 8 | | 3.2 | | dose |
| S.C. | dC*(P₂)T** | 4 | 2 | 2 | 1.6 | Pre and 0.1, 0.25, 0.5, | 72 hr post- |
| | dC(P₂)T | | 8 | | 6.4 | 1, 2, 4, 8, 24 and 48 hr | 1st dose |
| | | | | | | post 2nd dose | |

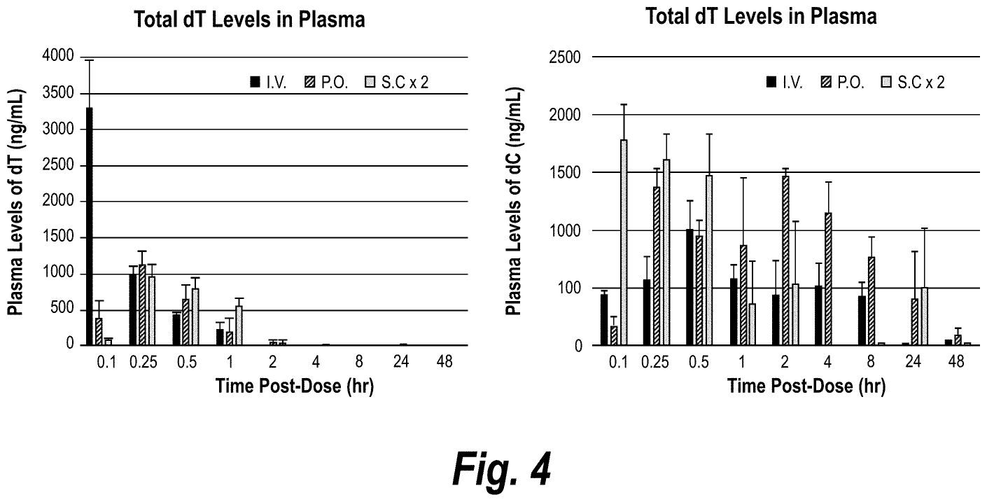

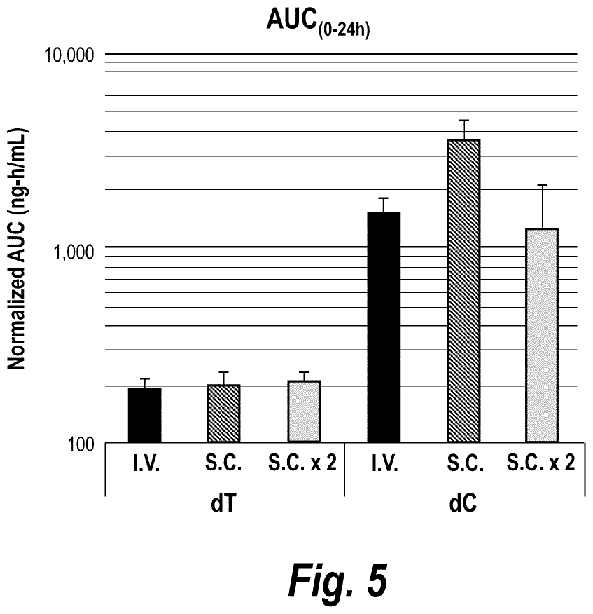

Plasma samples for the following analytes were analyzed: dT**, dC*, dT**MP, dC*MP, and dC*(P₂)dT**, and as with Example 1, only dT** and dC* were detectable. Plasma levels of label-normalized dT and dC levels following a single I.V. and one and two S.C. administrations of labeled and unlabeled dC(P₂)dT groups are shown in FIG. 4. FIG. 5 shows the plasma exposures for the two analytes for the three treatment groups for the initial 24 h post-dose, determined by calculating the mean area-under-the-curve (AUC₀₋₂₄h) for the total dose-normalized dT and dC plasma levels from FIG. 4. The mean AUC₀₋₂₄ₕ values among the three treatment groups are not statistically different, indicating the I.V. and S.C. administrations of dC(P₂)dT result in comparable exposures of dT and dC in plasma.

All publications, patents and patent applications cited in this specification are incorporated herein by reference for the teaching to which such citation is used.

The specific responses observed may vary according to and depending on the particular active compound selected or whether there are present carriers, as well as the type of formulation and mode of administration employed, and such expected variations or differences in the results are contemplated in accordance with practice of the present invention.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

### REFERENCES

Chansprasert, S. and Craigen W. J., (2017) Mitochondrial Disorders Causing Cardioskeletal Myopathies in Childhood. Cardioskeletal Myopathies in Children and Young Adults.
El-Hattab, A. W. and Scaglia, F., (2013) Mitochondrial DNA depletion syndromes: review and updates of genetic basis, manifestations, and therapeutic options. Neurotherapeutics. Apr:10(2):186-198.
Garone, C., Taylor, R. W., Nascimento, A., Poulton, J., Fratter, C., Domínguez-González, C., Evans, J. C., Loos, M., Isohanni, P.,Suomalainen, A., Ram, D., Hughes, M. I., McFarland, R., Barca E., Lopez Gomez , C., Jayawant, S., Thomas, N. D., Manzur, A. Y., Kleinsteuber, K., Martin, M. A., Kerr, T., Gorman, G. S., Sommerville, E. W., Chinnery, P. F., Hofer, M., Karch, C., Ralph, J., Cámara, Y., Madruga-Garrido, M., Domínguez-Carral. J., Ortez, C., Emperador, S., Montoya, J., Chakrapani, A., Kriger, J. F., Schoenaker, R., Levin, B., Thompson, J. L. P, Long, Y., Rahman, S., Donati, M. A., DiMauro. S., and Hirano M., (2018) Retrospective natural history of thymidine kinase 2 deficiency. J Med Genet. Aug; 55(8):515-521.
TK2-related mitochondrial DNA depletion syndrome, myopathic form: National Library of Medicine (US). Genetics Home Reference [Internet]. Bethesda (Md.): The Library; 2013 Sep; [reviewed 2013 Sept; cited 2013 Sep 19]; [about 6 screens]. Available from: https://ghr.nlm.nih.gov/condition/cystic-fibrosis

1. A method of treating mitochondrial DNA depletion syndrome in a subject in need thereof comprising administering to the subject a therapeutically effective amount of a multinucleotide composition comprising the compound of Formula I 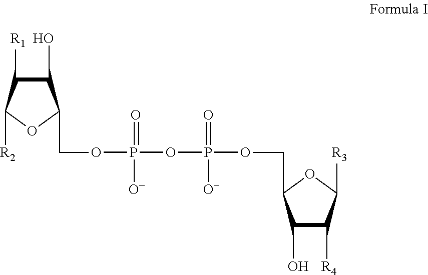
wherein:
R₁ is H or OH;
R₂ is a purine derivative or pyrimidine derivative;
R₃ is a purine derivative or pyrimidine derivative; and
R₄ is H or OH.

2. The method of claim 1, wherein the composition comprises the compound of Formula I wherein R₁ is H, R₂ is 5-methyl-2H-1λ2-pyrimidine-2,4(3H)-dione, R₃ is 4-amino-2H-1λ2-pyrimidine-2-one, and R₄ is OH.

3. The method of claim 1, wherein the composition is a mixture further comprising the compound of Formula I, wherein R₁ is OH, R₂ is 9λ2-purine-6-amine, R₃ is 2-amino-9λ2-purin-6(1 H)-one, and R₄ is OH.

4. The method of claim 1, wherein the composition is administered orally, enterically, intravenously, or subcutaneously.

5. The method of claim 1, wherein the composition is administered intravenously.

6. The method of claim 5, wherein blood plasma levels of each mononucleoside is higher after intravenous administration as compared to blood plasma levels after oral administration of individual mononucleotides.

7. The method of claim 1, wherein intravenous administration of the composition results in a blood plasma level having a first AUC₀₋₂₄ₕ and oral administration of individual mononucleotides results in blood plasma levels having a second AUC₀₋₂₄ₕ, and wherein the ratio of the first AUC₀₋₂₄ₕ to the second AUC₀₋₂₄ₕ is between about 100 to about 400.

8. The method of claim 1, wherein when the composition is administered at a dosage of between about 1 mg/kg to about 20 mg/kg, blood plasma concentration of the corresponding nucleosides is between about 50 ng/ml to about to about 5000 ng/ml.

9. The method of claim 1, wherein the mitochondrial DNA depletion syndrome comprises thymidine kinase 2 deficiency, succinyl CoA synthetase deficiency, deoxyguanosine kinase deficiency, succinyl CoA ligase deficiency, ribonucleotide-diphosphate reductase subunit M2 B enzyme deficiency, thymidine phosphorylase deficiency, and polymerase gamma deficiency.

10. The method of claim 9, wherein the mitochondrial DNA depletion syndrome comprises thymidine kinase 2 deficiency.

11. The method of claim 1, wherein the subject is a human.

12. A method for the treatment of thymidine kinase 2 (TK2) deficiency in a subject in need thereof comprising:
a) obtaining a nucleic acid sample from a subject;
b) determining if the subject has TK2 deficiency;
c) administering a therapeutically effective amount of a composition comprising a multinucleotide compound of Formula 1: 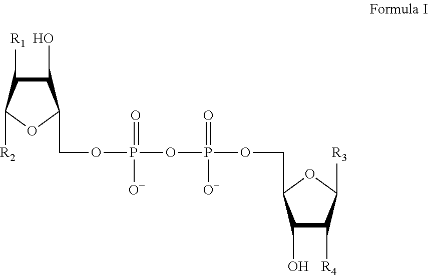
wherein:
R₁ is H or OH;
R₂ is a purine derivative or pyrimidine derivative;
R₃ is a purine derivative or pyrimidine derivative; and
R₄ is H or OH; and
d) measuring blood plasma levels of corresponding mononucleosides;
wherein the blood plasma levels of corresponding nucleosides is between about 50 ng/mL to about 5000 ng/mL.

13. A compound of Formula I: 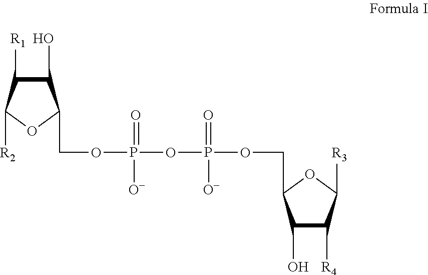
wherein
R₁ is H or OH;
R₂ is a purine derivative or pyrimidine derivative;
R₃ is a purine derivative or pyrimidine derivative; and
R₄ is H or OH.

14. The compound of claim 13, wherein the compound is substantially free from impurities.

15. The compound of claim 13, wherein when R₁ is H, R₂ is thymine and R₃ is cytosine, R₄ is OH.

16. A pharmaceutical composition comprising at least one pharmaceutically acceptable excipient and a therapeutically effective amount of the compound of claim 13.

17. The composition of claim 16, wherein the compound comprises R₁ is H, R₂ is 5-methyl-2H-1λ2-pyrimidine-2,4(3H)-dione, R₃ is 4-amino-2H-1λ2-pyrimidine-2-one, and R₄ is OH.
